(19) United States
(12) Patent Application Publication
(10) Pub. No.: US 2015/0202404 A1
(43) Pub. Date: Jul. 23, 2015

(54) VALVE ARRANGEMENT AND A REBREATHING SYSTEM COMPRISING SAID VALVE ARRANGEMENT

(71) Applicant: OXYRED AB, Stockholm (SE)

(72) Inventors: Ola Patriksson, Karlstad (SE); Jan Stööd, Hammaro (SE); Hans Örnhagen, Tungelsta (SE)

(73) Assignee: OXYREB AB, Stockholm (SE)

(21) Appl. No.: 14/423,137

(22) PCT Filed: Aug. 30, 2013
(86) PCT No.: PCT/SE2013/051022

§ 371 Date: Feb. 23, 2015

(30) Foreign Application Priority Data
Aug. 30, 2012 (SE) 1250965-9

(51) Int. Cl. A61M 16/20; A61M 16/06; A61M 16/22; A61M 16/00

(52) U.S. Cl. CPC A61M 16/204; A61M 16/0045; A61M 16/06; A61M 16/22; A61M 2202/0208; A61M 2202/0225; A61M 2205/3334; A61M 2205/44

(57) ABSTRACT

This invention relates to a valve arrangement (8, 18) arrangeable to be used in a rebreathing system (1), said rebreathing system (1) comprising an oxygen supplying member (5, 15), a breathing mask (4, 14), a gas reconditioning unit (3, 13) where carbon dioxide in the exhaled gas is absorbed, a counter lung (2, 12) and a breathing passage (42, 142), wherein said valve arrangement (8, 18) is arrangeable to be used in a back and forth (two way?) breathing passage (42, 142) between said counter lung (2, 12) and said breathing mask (4, 14) of said rebreathing system (1), said valve arrangement (8, 18) containing an oxygen supply arrangement arranged to supply oxygen to the breathing passage (42, 142) provided that a predetermined level of oxygen pressure is exerted on said supply arrangement from said oxygen supplying member (5) and arranged to close the breathing passage when said oxygen supply pressure is below a predetermined level. The invention also relates to a rebreathing system (1) comprising said valve arrangement (8, 18).

### FIELD OF THE INVENTION

The present invention relates to a valve arrangement arrangeable to be used in a rebreathing system, said rebreathing system comprising an oxygen supplying member, a breathing mask, a gas reconditioning unit where carbon dioxide in the exhaled gas is absorbed, a counter lung and a breathing passage. The invention also relates to a rebreathing system comprising said valve arrangement.

### BACKGROUND INFORMATION

A person is breathing primary to eliminate carbon dioxide and provide the metabolism with oxygen. The cerebral control system regulating ventilation is aimed to maintain a constant pH of 7.4 in the arterial blood. With the buffers in the blood this is reached at a partial pressure of 5.5 kPa of CO₂ in the alveolar gas. An increase of metabolism and delivery of CO₂ to the blood and lungs, as during muscle work, increases the PCO₂ and decreases the pH, which signals to the respiratory center to increase the alveolar ventilation so the CO₂ concentration and pH can be brought back to the desired levels. The surrounding air is almost free from CO₂ and consists of about 20% of oxygen. At each inhalation the body extract about 5% units of that oxygen and the remaining 15% of oxygen is exhaled to the atmosphere again together with CO₂ which is about 5% of the volume exhaled. This means that only a quarter of the available oxygen in the air is used for metabolism. To reduce the amount of gas needed in a breathing equipment, and make it possible to reuse the oxygen exhaled, closed circuit breathing apparatus also called rebreathers are used. In a rebreather the produced CO₂ is absorbed in a scrubber, most often calcium hydroxide. Rebreathers can also be used to provide high oxygen fractions for medical purposes without wasting a lot of oxygen. Unfortunately there is a lethal risk if the oxygen supply stops while the scrubber is still effective since no dyspnea (air hunger) is felt as long as the CO₂ is eliminated. Lack of oxygen is a very weak and slow stimulus to the breathing system and there will be no acute warning signal to the wearer before the unconsciousness is a fact.

WO 99/13944 discloses a diving equipment with sensor means that detect the oxygen content of the exhaled gas and means for injecting oxygen into the exhaled gas to reinstate the oxygen content so as to lie within a desired range for rebreathing. If there is a deviation from the desired range an alarm indicator is triggered alerting the diver to the situation and allowing her to decide which action to take.

U.S. Pat. No. 4,141,353 discloses a breathing apparatus for divers based on the physiological relation between oxygen extraction and ventilation. When the volume ventilated has reached a given quantity, respiratory gas, stored in a bottle, is injected to the respiratory circuit. The excess volume is vented to the surroundings and a new breathing period is then started simultaneously as the bottle is filled once again with respiratory gas. The breathing apparatus comprises a warning valve that gives an increased breathing resistance if there has been no addition of oxygen rich supply gas to the breathing loop. This is to warn the user to take necessary action.

### SUMMARY OF THE INVENTION

It is an object of the present invention to overcome or at least minimize at least one of the drawbacks and disadvantages of today available systems for delivery of oxygen to closed breathing systems. This can be obtained by said valve arrangement is arrangeable to be used in a two way breathing passage between said counter lung and said breathing mask of said rebreathing system, said valve containing an oxygen supply arrangement arranged to supply oxygen to the breathing passage provided that a predetermined level of oxygen pressure is exerted on said supply arrangement from said oxygen supplying member and arranged to close the breathing passage when said oxygen supply pressure is below a predetermined level, and a breathing system comprising said valve arrangement.

The invention prevents the user to inhale breathing gas where the CO₂ has been absorbed but no oxygen has been added which otherwise may cause the user to suffer from hypoxia that in the worst may be lethal.

According to one aspect of the invention, the valve arrangement comprise an oxygen receiving chamber provided with an oxygen inflow and an oxygen outflow having an oxygen flow restrictor. A spring biased protective valve is in fluid communication with said oxygen receiving chamber, said oxygen supply pressure contained therein acting on said protective valve to set the protective valve in an open position.

According to another aspect of the invention, the spring biased protective valve is regulated to open at a pressure that is significantly lower than the oxygen supply pressure.

According to still another aspect of the invention, said spring biased protective valve comprises a breathing passage closing member co-acting with a seat in a valve arrangement housing, said seat preferably being an integrated part of a connecting device between the breathing mask and said other parts of the rebreathing system wherein a compact design is achieved.

According to a further aspect, said oxygen supplying source supplies oxygen having an oxygen pressure in the range of 100-300 kPa, preferably 200-300 kPa.

According to a further aspect, the breathing system comprises a heat exchanger being arranged to absorb heat generated by the CO₂-absorbing reaction in the gas reconditioning unit wherein a more pleasant breathing is obtained. The gas reconditioning unit is arranged at a relative angle to the extension of the heat exchanger wherein the volume of a compartment within the heat exchanger is minimised in order to minimise the volume of breathing gas not passing the gas reconditioning unit and providing a large contact area for the breathing gas in order to obtain efficient cooling. A large contact area for the breathing gas is obtained in that the heat exchanger constitutes a major part of the wall enclosing said compartment.

In order to secure free passage of surrounding air to and from the mask when the protective valve is in a closed position, said breathing mask comprises an overpressure valve and an air inflow valve. According to a further aspect said air inflow valve comprises a whistle.

According to yet another aspect of the invention, the oxygen supplying source is a pressurized oxygen gas source or a chemically bound oxygen source whereby the oxygen source may be selected dependent on the intended application.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a side view in a cross section of a rebreathing system according to the invention,

FIG. 2 shows a front view of a rebreathing system according to the invention,

FIG. 3 shows a cross section view of a valve arrangement according to the invention,

FIG. 4A shows the registration of mask O₂ and CO₂ fractions when using a rebreathing system with an oxygen supply,

FIG. 4B shows the registration of mask O₂ and CO₂ fractions when using a rebreathing system without an oxygen supply,

FIG. 5 shows an alternative embodiment in a cross section side view, and

FIG. 6 shows an alternative embodiment in a cross section side view.

### DETAILED DESCRIPTION OF THE PREFERRED EMBODIMENTS

The following detailed description, and the examples contained therein, are provided for the purpose of describing and illustrating certain embodiments of the invention only and are not intended to limit the scope of the invention in any way.

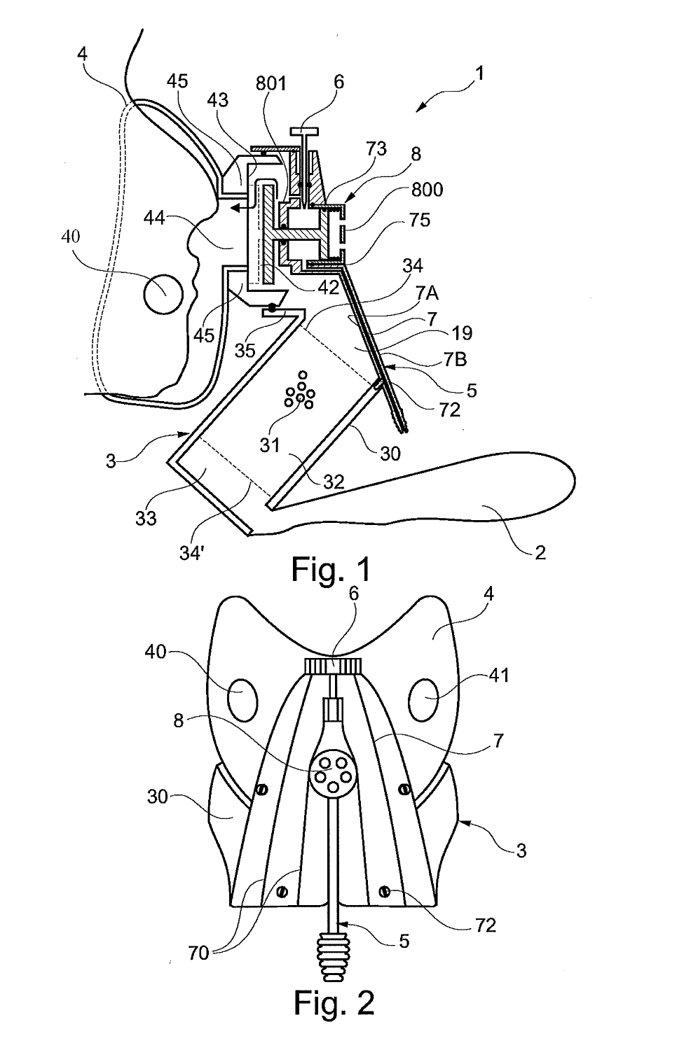

FIG. 1 is a cross section side view of a rebreathing system 1 in a preferred embodiment according to the invention. The rebreathing system 1 comprises a breathing mask 4 covering the nose and mouth of a person, an oxygen supplying member 5, a gas reconditioning unit 3, a counter lung 2, a valve arrangement 8, a flow restrictor 6 and a heat exchanger 7.

The breathing mask 4 is connected to said gas reconditioning unit 3 which in turn is connected to said counter lung 2 creating a breathing passage 42 that allows breathing gas to flow between the breathing mask 4 and the counter lung 2 through said gas reconditioning unit 3. The gas reconditioning unit 3 comprises a canister 30 filled with scrubbing material 31, (e.g. Ca(OH)₂, LiOH, NaOH or KOH), that absorbs the CO₂ produced.

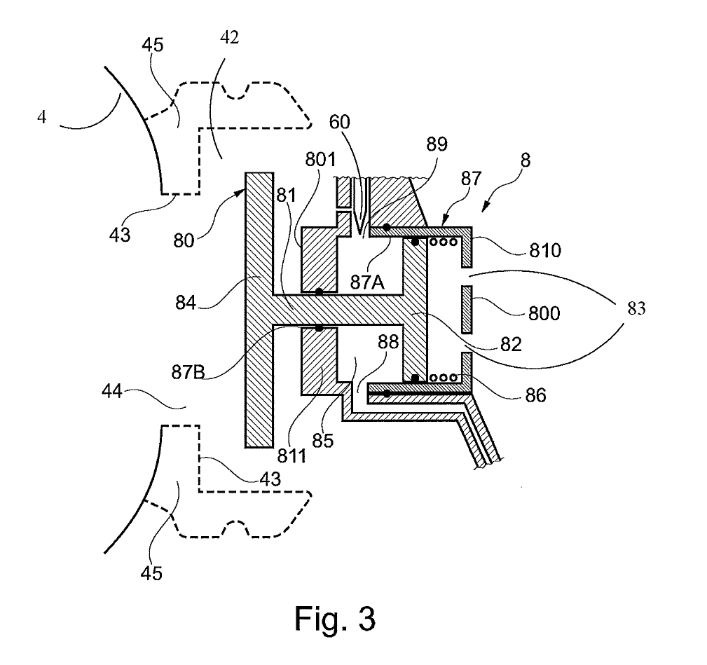

The rebreathing system 1 furthermore comprises a valve arrangement 8, said valve arrangement 8 having an inflow port 88 and an outflow port 89 (see FIG. 3) for supplying oxygen to the breathing passage. The valve arrangement 8 will be described in detail further on. The oxygen is supplied to the rebreathing system 1 from an external oxygen source (not shown) e.g. an oxygen bottle via an oxygen supplying member 5 in fluid communication with the valve arrangement 8.

When using the rebreathing system 1, exhaled breathing gas flows from the breathing mask 4, into the breathing passage 42 comprising the gas reconditioning unit 3 in which CO₂ is absorbed in the scrubbing material in the canister 30 during development of heat and into the counter lung. On inhalation, a return flow of breathing gas is created from the counter lung, through the reconditioning unit 3 and back to the breathing mask 4. The breathing gas passes the heat exchanger 7 arranged in the breathing passage between the breathing mask 4 and the reconditioning unit 3 where at least some of the generated heat in the canister 30 is cooled off in order to lower the temperature of the breathing gas to a pleasant temperature. Via the valve arrangement 8, oxygen is supplied to the breathing gas.

The counter lung 2 is arranged at a lower end 33 of said canister 30 e.g. the counter lung 2 is attached, eg. in a groove, on said canister 30 and sealed by a clamp or other sealing means (not shown). The skilled man realises that other options for attachment are possible. The canister 30 comprises an inner space 32 where the scrubbing material 31 is arranged between a first 34 and a second 34′ perforated partition. At an exhalation, breathing air passes through the first perforated partition 34 and into the inner space 32 where the scrubbing material 31 absorbs CO₂. The breathing gas passes on through the second perforated partition 34′ and into the counter lung 2. The perforations in the partitions 34, 34′ are preferably located so that breathing air is uniformly distributed through the scrubbing material in order to increase the efficiency of the CO₂ absorption and increase the lifespan of the scrubbing material, e.g. avoid channeling. At an inhalation, the breathing gas flows the opposite way from the counter lung 2 through the canister 30 to the breathing mask 4. Accordingly, absorption of CO₂ takes place twice, during both exhalation and inhalation which further increase the efficiency. Hence, the canister may be kept small in size which improves the comfort during use.

The valve arrangement 8 comprises a cylindrical protective valve housing 87, having an outer end 800 which cooperates with a, preferably integrated, part of the heat exchanger 7 forming an inner end 801 of said valve housing 87 (see FIG. 3). The inner end 801 is placed inside the breathing passage 42 and the outer end faces open air. The protective valve housing 87 also comprises an oxygen inflow port 88 and an oxygen outflow port 89 arranged in the cylindrical wall of the protective valve housing 87 adjacent the inner end 801 of the valve arrangement 8 for continuous supply of oxygen to the breathing passage 42. A spring 86 biased protective valve 80 is arranged with the valve arrangement 8. The protective valve housing 87, i.e. valve cylinder, accommodates a piston 82 connected to a valve disc 84 at the protective valve 80 via a piston rod 81 passing, in an airtight (sealed) manner, a through hole 87 B of an inner end wall 811 of the protective valve housing 87. The piston 82 is arranged in sealed abutment to the inside 87A of said protective valve housing 87. The outer end wall 810 is provided with at least one passage/opening 83 to open air in order to make movement of the spring biased protective valve 80 possible. The valve disc 84 is arranged to co-act with a seat 43 (see FIG. 1) arranged in the breathing passage 42, between the breathing mask 4 and the canister 30 in order to close the breathing passage 42 between the breathing mask and the counter lung 2. The seat 43 may preferably be integrated in (form part of) a connecting device 45 between a breathing air opening 44 in the mask 4 and the breathing passage 42. The valve arrangement 8 is further mounted in the upper part of the heat exchanger 7.

The breathing passage 42 includes a heat exchanger 7, which in the preferred embodiment constitutes an integrated part of the wall of the breathing passage 42. In a preferred embodiment, the heat exchanger comprises the forward facing wall of the breathing passage 42 (from a user's perspective) in order to have best cooling effect. The heat exchanger 7 is connected to the mask 4, preferably via the connecting device 45, at its upper end. The heat exchanger 7 is preferably made of a 3 mm copper plate, or a plate made of another material having high thermal conductivity, arranged with cooling flanges 70 at the outside of the copper plate (see FIG. 2), the cooling flanges being substantially perpendicularly attached at the copper plate e.g. by soldering. The heat exchanger 7 preferably acts like a holder for the valve arrangement 8 and oxygen supply line 5. The upper end of the canister 30 is at its' outer side connected, preferably by screws 72, at the lower end of the heat exchanger 7. The canister is connected to the inside 7A of the copper plate 7 and at an relative angle to the extension of the copper plate thereby creating a compartment 19 in the breathing channel where breathing air passing to or from the canister is directed towards the copper plate. The perforations in the partition may be designed to contribute to a proper distribution of the air flow, e.g. by arranging the perforations at a suitable angle through the partition. The inner side of the upper end 35 of the canister is connected to the mask 4 via the lower end of the connecting device 45. All connections are sealed in an airtight manner to prevent leakage in or out of the breathing system. The copper plate is preferably slightly curved outwards (convex) in order to provide sufficient room inside the breathing passage 42 for the valve arrangement 8 and also to provide a sufficient flow passage, i.e. not causing too much flow restriction, for breathing air. The volume of the compartment 19 within the copper plate (dead space) shall however be as small as possible, preferably max 0.2 l, in order to force as much breathing air as possible to pass the reconditioning unit before being inhaled again. The copper plate in combination with an angled arrangement of the reconditioning unit 3 contributes to reduce the volume while still providing efficient cooling. The skilled person realizes that the heat exchanger may be designed in another manner and still provide the desired effect.

The valve arrangement 8 is arranged in the upper end of the heat exchanger 7, in a through hole 73 in the heat exchanger 7 with the outer end 800 placed on an outer side 7B of the heat exchanger 7 and the inner end 801 placed on the inner side 7A of the heat exchanger 7, inside the breathing channel 42, the inner part 84 of the piston being arranged to co-act with an opening in the mask 4 for opening and closing of a breathing air passage in to the mask 4. As earlier described, the valve arrangement 8 comprises an outlet port 89 for supply of oxygen into the breathing passage. In connection to this outlet port 89, a flow restrictor 6 is arranged, preferably a set screw 6 where the point 60 of the set screw 6 is arranged to co-act with the outlet port 89 in the valve arrangement 8 for adjustment of the oxygen supply. The set screw may be arranged in a threaded hole 74 in the heat exchanger 7 and may be provided with a scale for convenient adjustment of the oxygen supply. The oxygen supplying member 5, preferably a copper pipe, extends on the outer side 7B of the heat exchanger 7 and may be connected to an inflow duct in the outer end 800 of the protective valve housing 87. More preferred, the oxygen supplying member 5 is led through a hole 75 in the heat exchanger 7 to the inflow port 88 in the inner end 801 and is attached thereto, preferably by soldering.

FIG. 2 is a perspective front view slightly from above of a rebreathing system 1 according to the invention. The breathing mask 4 is at least designed to cover the nose and mouth of the wearer and extend along the cheeks and chin to secure tight contact. A variant that also covers the eyes and seals against the forehead (a full face mask) is also conceivable in order to avoid smoke or other gas to come into the eyes, which forces the person to shut the eyes, thereby making orientation and evacuation difficult. The mask 4 comprises an overpressure valve 40 and an air inflow valve 41, both are preferably spring loaded and positioned at the side of the mask in order to secure free passage of air to and from the mask 4 when the protective valve 80 is closed. The heat exchanger 7 act as mounting device for the flow restrictor 6, the valve arrangement 8 and the oxygen supplying member 5. The canister is mounted at the lower end of the connecting device 45 in an relative angle to the copper plate as earlier described. The lower end of the copper plate is mounted at the upper end of the canister, eg. via a screw 72. The copper plate have a length that is sufficient in order for the canister 30 to be positioned below the users chin, preferably in front of the throat and above the chest, making the rebreathing system 1 more comfortable and more stable to wear, and also less bulky. The heat exchanger preferably has a somewhat triangular shape, having a base width in the order of the width of a face, thereby providing a large cooling area in the region where the canister is mounted. At its' upper end, in the region where the valve arrangement 8 is mounted, is shall be formed to fit snugly in front of the opening in the breathing mask 4 in order not to become a sight obstacle.

With reference to FIG. 3, the use of the rebreathing system 1 will now be described. To use the rebreathing system 1 described above, the user places the breathing mask 4 over the nose and mouth and the mask is held in place in a known manner e.g. by an elastic harness around the user's head. When the external oxygen source is activated, preferably having a supply pressure of 100-300 kPa, more preferred 200-300 kPa above ambient pressure, the oxygen flows through the oxygen supplying member 5 and into a first chamber 85 in the protective valve housing 87 via the inflow port 88. The point 60 of the flow restrictor 6 in the outflow port 89 act as a restriction valve, causing the pressure to increase in the first chamber 85 while, at the same time, allowing a certain amount of oxygen to flow into the breathing passage via the outflow port 89. The supply of pressurized oxygen to the first chamber 85 forces the valve arrangement 8 to its' open position since the spring biased protective valve 80 is regulated to open at a pressure that is significantly lower than the oxygen supply pressure, preferably in the magnitude of about 2-10 times lower, or in the range of 30-40 kPa above ambient pressure. As the protective valve 80 is forced to move towards its' outward position, the valve disc 84 of the protective valve 80 leaves its seat 43 and opens up the breathing air opening between the breathing mask 4 and the breathing passage 42. The flow restrictor 6 is preferably adjusted to let an oxygen flow of 1-1.5 l/minute to pass the outflow port 89, which is 3 to 4 times the metabolic need in rest.

During breathing, exhaled breathing gas flows into the breathing passage 42 via the breathing air opening 44 in connection of the seat 43 of the valve arrangement 8, passes the heat exchanger 7 and flows into the canister 30 and to the counter lung 2. The CO₂ is absorbed by the scrubbing material in the canister 30 during development of heat. When the user inhales, the breathing gas flows the opposite way from the counter lung 2 via the canister 30, passing the heat exchanger 7 which lowers the temperature on the breathing gas and flows back into the breathing mask 4 through the breathing air opening 44 in connection to the protective valve seat 43. Irrespective if the user makes an inhalation or exhalation the overpressure in the first chamber 85 ensures a continuous oxygen supply to the breathing passage 42. Since the oxygen supply is greater than the metabolism, the counter lung 2 will be filled to a maximum at the end of an exhalation and an overpressure is built up in the breathing system. This overpressure will open the overpressure valve 40 and the redundant gas leaks out in the surroundings, thanks to this overpressure valve 40 a comfortable breathing is allowed and it prevents mask leakage. This “ventilation” of the breathing circuit, through the overpressure valve 40, will ventilate nitrogen from the body to the surroundings and prevents a nitrogen built-up even if the user is loaded with nitrogen after a previous dive, this in the case when the rebreathing system 1 is used in a diving application.

When the oxygen supply ends or is reduced, the pressure in the oxygen supplying member 5 and the first chamber 85 will be reduced. The cylinder volume of the first chamber 85 will be decompressed to atmospheric pressure due to lack of oxygen supply pressure and the protective valve 80 moves by force from the spring 86 back to its starting positioning, i.e. the valve disk 84 of the protective valve 80 again rests against its seat 43. This closes the breathing passage 42, preventing the user to inhale breathing gas where the CO₂ has been absorbed but no oxygen has been added which otherwise may cause the user to suffer from hypoxia that in the worst may be lethal.

If the valve arrangement 8 closes due to lack of oxygen supply, inspiration causes a relative under-pressure in the mask 4, which opens the air inflow valve 41 at a relative under-pressure of −0.5 kPa. The following expiration causes a relative overpressure in the mask 4 which causes the outflow/overpressure valve 40 to open at +0.5 kPa and the exhaled air with CO₂ to leave the body. The air inflow valve 41 preferably comprises a whistle (not shown) causing a whistle sound at every inhalation and/or exhalation which alerts the user that there is no oxygen supply to the rebreather and action has to be taken. This whistle function may also be of decisive importance when the rebreathing system 1 is used on an unconscious person that does not notice the sound, making surrounding people observant and exchange the oxygen supply.

A volume of 160 ml of scrubbing material in the canister 30 is sufficient for use of the rebreather during one hour for a person in rest having a weight of 90 kg. The canister 30 and the amount of scrubbing material are preferably dimensioned after desired action time and the amount of the oxygen supply. There are different ways to exchange/refill the canister 30 with scrubbing material 31. For example, the first perforated partition 34 close to the counter lung 2 is preferably detachably arranged e.g. by means of some kind of snap-in member at the inner wall of said canister 3. Alternatively, a bottom part of the canister housing comprising the first perforation partition 34 may be detachably arranged at the lower end of the canister housing.

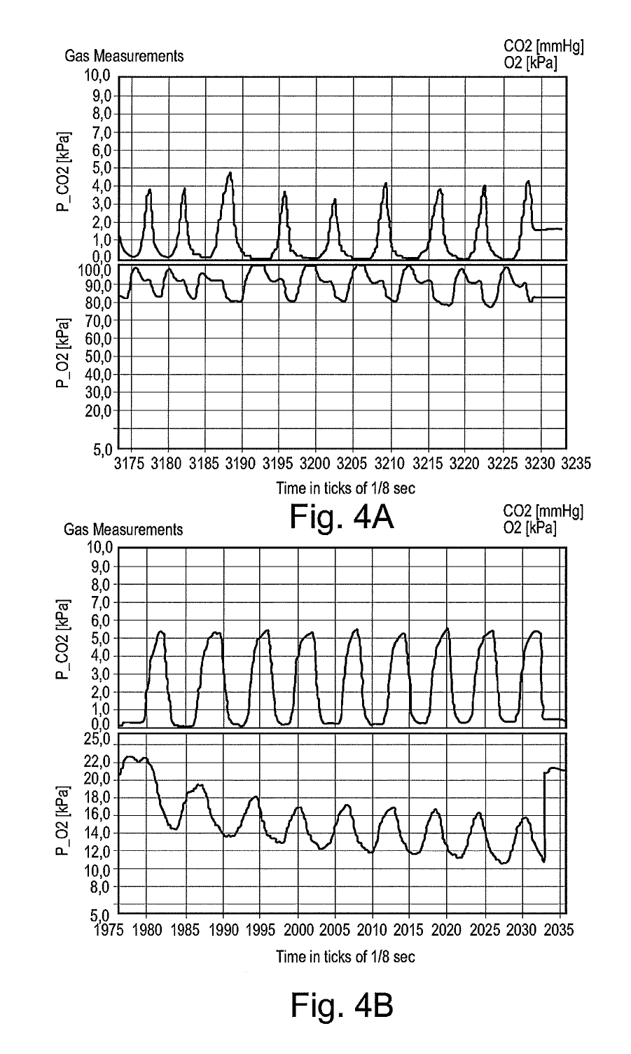

In FIG. 4 two graphs are shown. FIG. 4 A shows the registration of oxygen and CO₂ in the breathing gas when using the rebreathing system 1 with an oxygen supply. FIG. 4 B shows the registration of oxygen and CO₂ in the breathing gas when using the rebreathing system 1 without an oxygen supply in order to exemplify what happens in a conventional rebreather system, i.e. without a valve arrangement according to the invention, when the oxygen supply ends or malfunctions. The upper curve in both FIGS. 4 A and 4 B shows the CO₂ curve and the lower curve in both FIGS. 4 A and 4 B shows the oxygen curve. At a normal use of the rebreathing system 1 with an oxygen supply (as shown in FIG. 4 A) both curves are stable, showing a regular variation of the partial pressure of both CO₂ and O₂ depending of the breathing cycle. In FIG. 4 B the applicant was testing the rebreathing system 1 without an oxygen supply. The lower oxygen curve clearly shows how the partial pressure of oxygen gradually are reduced in connection to each breath but the user does not suffer from dyspnoea since the CO₂ is absorbed by the scrubbing material. The user will eventually suffer from hypoxia that in the worst may be lethal. In table 1 below is further shown the result from performed tests.

TABLE 1

| | | Estimated | Measured | CO2 | | | | | | | | |
|---|---|---|---|---|---|---|---|---|---|---|---|---|
| Canister | | CO2 | oxygen | absorbing | CO2 | time | CO2 | time | CO2 | time | CO2 | Absorption |
| volume | | production | flow | media | absorbing | to 1% | absorption | to 2% | absorption | to 3% | absorption | capacity |
| (ml) | Activity | (ml/min) | (ml/min) | (type/name) | media (ml) | (s) | (min/g) | (s) | (min/g) | (s) | (min/g) | (l CO2/kg) |
| A | Sitting | 200 | | Spherasorb | 118 | 60 | 0.51 | | | 75 | 0.64 | 102 |
| A | Sitting | 200 | | Spherasorb | 112 | | | 58 | 0.52 | | | |
| A | Sitting | 200 | | Spherasorb | 114 | 58 | 0.51 | 67 | 0.59 | | | 102 |
| A | Sitting | 200 | | Spherasorb | 120 | 80 | 0.67 | 85 | 0.71 | | | 133 |
| A | Sitting | 200 | | Sofnolime | 108 | 59 | 0.55 | 70 | 0.65 | | | 109 |
| A | Sitting | 201 | | Sofnolime | 114 | 75 | 0.66 | 80 | 0.7 | 83 | | 132 |
| B | Sitting | | | Sofnolime | 138 | 111 | 0.8 | 126 | 0.91 | 138 | | |
| B | Sitting | 260 | 325 | Sofnolime | 142 | 35 | 0.25 | 67 | 0.47 | 80 | 0.56 | |
| | and walking | | | | | | | | | | | |
| B | Sitting | | | Sofnolime | 140 | 97 | 0.69 | 122 | 0.87 | | | |

Canister A, Volume 140 ml,
Canister B, Volume 160 ml
Spherasorb ®,, Mesh size 2.0-4.0 mm.
Sofnolime ®,, Mesh size 1.0-2.5 mm.

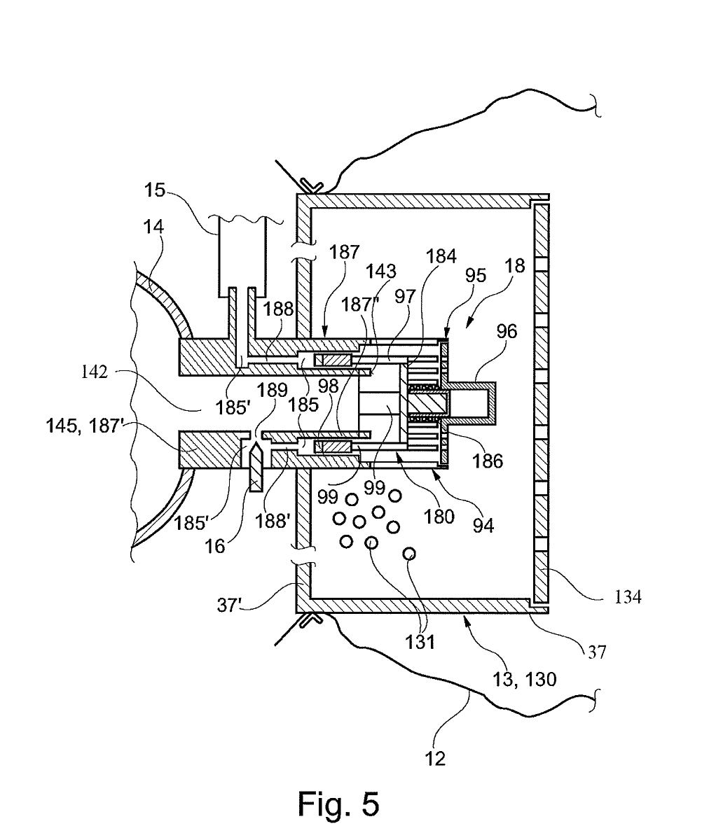

In FIG. 5 is shown, in a cross section, an alternative embodiment of the rebreathing system 1 according to the invention. For ease of understanding, features whose function is the same or basically the same as those earlier described are identified by the same reference numbers although being prefixed by the number “1”, e.g. the common feature “breathing bag 2” is identified as “breathing bag 12”, the “canister 3” is identified as “canister 13” etc.

The rebreathing system 1 comprises a breathing mask 14, a gas reconditioning unit 13, and a counter lung 12, The breathing mask 14 is connected to said gas reconditioning unit 13 which in turn is connected to said counter lung 12 creating a breathing passage 142 that allows breathing gas to flow between the breathing mask 14 and the counter lung 12 through said gas reconditioning unit 13. The gas reconditioning unit 13 comprises a connecting device 145 and connected thereto; a valve arrangement 18, a canister 130, an oxygen supplying member 15 and an oxygen flow restrictor 16.

The mask 14 is connected to the inner end of the connecting device 145 and the valve arrangement 18 is connected to the outer end of the connecting device 145. In this embodiment, the connecting device 145 has the form of a pipe. Preferably, the wall 187′ of the connecting device 145 forms an integrated part of the cylindrical housing 187 of the valve arrangement 18. The valve arrangement 18, which will be described in detail later, is arranged to open or close the breathing passage 142. The canister 130, which may have a cylindrical form comprising an inner end wall 37′, a cylindrical wall 37 and an outer end wall 134, contains scrubbing material 131 (e.g. Ca(OH)₂, LiOH, NaOH or KOH) that absorbs the CO₂ produced. The outer end wall 134 forms a partitioning wall and is removably attached to the canister 130 so that the scrubbing material 131 may be exchanged in a simple manner.

The canister 130 encapsulates the valve arrangement 18. The valve arrangement 18 is embedded in scrubbing material 131. The counter lung 12 is attached to the canister 130, preferably at the inner end of the cylindrical wall 37 of the canister 130. At least one of, but preferably both of, the cylindrical wall 37 and the outer end wall 134 of the canister 130 is perforated with e.g. holes or slits, allowing breathing gas to pass to and from the counter lung 12. The canister 130 is connected to the outside of the cylindrical housing 187 of the connecting device 145 via a through hole in the centre of the inner end wall 37′ where the outer end of the connecting device 145 and the valve arrangement 18 is inserted into the canister 130.

The inner end wall 37′ forms a bottom at the inner side of the canister 130. At the bottom, the cylindrical housing 187 is centrally positioned. The outer end of the cylindrical housing 187 comprises a slit portion 94 with an upper edge 95. A lid 96 is arranged at the upper edge 95, said lid preferably being perforated. Said slit portion and preferably also said lid forms a passage for breathing air between the mask 14 and the canister 130.

A protective valve 180 is movably arranged inside said cylindrical housing 187. The protective valve 180 comprises an inner cylindrical part 97 arranged with a gasket 98 at the inner edge. The cylindrical part 97 comprises a closed outer wall 184. The inner cylindrical part 97 is spring biased, said spring resting against the inside of lid 96 and the outside of the wall 184. The cylindrical part 97 is provided with oblong through holes 99 which preferably are symmetrically arranged along the cylindrical part 97 for passage of breathing gas.

The inner end of the cylindrical housing 187 is double walled thereby forming a trench 185 wherein the cylindrical part 97 is arranged to move between an inner closed position and an outer open position. An inner wall 187″ of the cylindrical housing 187 comprises a seat in the form of a gasket 143 at its rim which seals against the outer wall 184 when the protective valve 180 is in the inner closed position. At the bottom of said trench 185 two vents 188, 188′ are arranged and said vents 188, 188′ communicate with a circumferential oxygen supply chamber 185′ in the cylindrical wall 187′ of the connecting device 145.

The oxygen supply chamber 185′ is connected to the external oxygen supply by an oxygen supplying member 15. In the oxygen supply chamber 185′ there is also arranged an adjusting hole 189 that forms a passage between the oxygen supply chamber 185′ and the breathing passage 142. By means of a oxygen flow restrictor 16 the oxygen flow to the breathing passage 142 can be controlled. The oxygen flow restrictor 16 also acts as a throttle valve adjusting the overpressure in said oxygen supply chamber 185′ which is transferred to the trench 185 via the two vents 188, 188′.

The oxygen that flows into the trench 185 creates an overpressure that is bigger than the force from a spring 186, thereby causing the protective valve 180 into an open position. In the open position breathing gas may flow in both directions in the breathing passage, i.e. from the breathing mask 1 into a passage in the centre of the cylindrical housing 187, therefrom passing through the oblong through holes 99 in the protective valve 180 into the canister and via the perforations in the canister wall 37, 134 further into the breathing bag 12, and vice versa. Irrespective of the user makes an inhalation or exhalation the overpressure in the trench 185 secures an open breathing passage 142 wherein oxygen is continuously supplied to the breathing air via the adjusting hole 189.

When oxygen pressure in the oxygen supply falls below a predetermined level, the overpressure falls simultaneously and the protective valve 180 is forced into the closed position by the force from the spring 186, i.e. the closed outer wall 184 of the protective valve 180 again rests against the gasket 143. In the closed position, flow of breathing gas in the breathing passage is prevented since no breathing gas can pass through the oblong holes 99 in the protective valve 180. Hypoxia is thereby prevented.

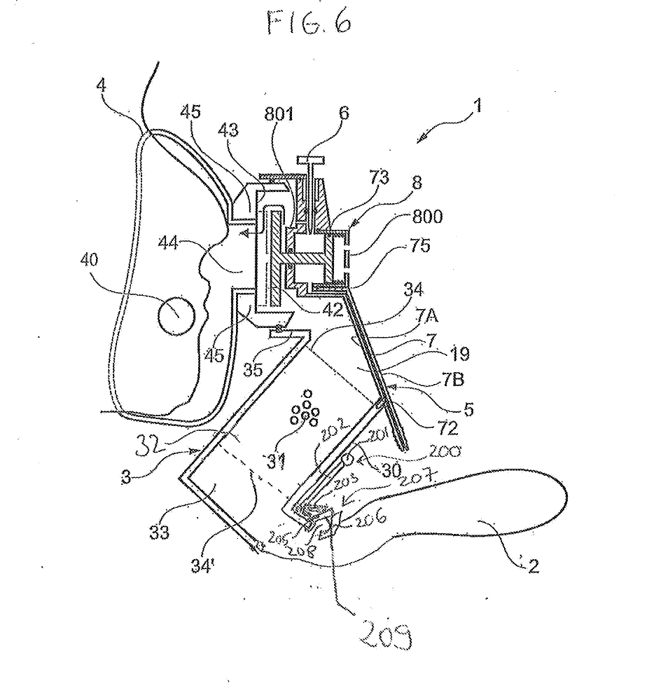

FIG. 6 shows an alternative embodiment of the rebreathing system 1 according to the invention. The rebreathing system 1 is the same as described in accordance with the earlier figures except for an additional mix valve 207. The mix valve 207 is controlled by a, preferably mechanical, control device 200, said control device comprises a knob 201 and a stick 202. The control device 200 preferably extends along the outer of the canister 30. The knob 201 is preferably arranged at un upper end of the stick 202 and the lower end of the stick 202 is arranged to interact with the mix valve 207. The mix valve 207 comprises a pivot able lid 209, attached adjacent an opening 208 into the interior 2 of the rebreathing system 1. The lid 209 is attached by means of a hinge 205 and interacts with a seating at a guide wall 206, to enable movement between a fully closed opening 208 and an adjustable amount of through passage of the opening 208. An adjusting mechanism 203 is connected between said stick 202 and said lid 209 to facilitate adjustable positioning of the lid. By maneuvering the stick 202 (e.g. turning, or pivoting), preferably by grasping a knob 201, the adjusting mechanism 203 may move the lid 209, i.e. the control device 200 may easily be moved between a closed (high oxygen) and an open (low oxygen) state. When the control device 200 is in a closed state (high oxygen) the rebreathing system 1 is a fully closed circuit and the air passage 208 in the mix valve 207 is closed and no air enters the rebreathing system 1 from the outside/surroundings. The oxygen flow from the outflow port 89, in the valve arrangement 8, to the rebreathing system 1 may normally be 1-1.5 l/minute. When the control device 200 is in the closed state the oxygen saturation is at a maximum, as close to 100% as possible. This state is appropriate to use for example at decompression illness, smoke accidents, cluster headache and high altitude sickness. However, in some situations it may be desired to avoid maximum oxygen saturation, which is easily arranged for by means of the control device 200.

When the control device 200 is in an open (low oxygen) state the rebreathing system 1 is a semi closed circuit. Preferably, also in this state the oxygen flow into the rebreathing system 1 from the outflow port 89 may be 1-1.5 l/minute. When the control device 200 is moved to the open state the hinge opens up the air passage 208 and allows outside air to enter the rebreathing system 1. The breathing gas in the counter lung 2 will in this state be mixed with normal air (21% O₂/78% N₂) entering from the now open mix valve 207. In the open state the oxygen saturation may be between 21-50%. This state is preferably appropriate for everything that not needs a 100% oxygen saturation.

An beneficial aspect with this solution is the ability to choose the oxygen saturation as required. Most often a 100% oxygen saturation is not needed so with this solution a lower saturation of oxygen is available but at the same time the oxygen flow from the outflow port 89 is retained at 1-1.5 l/minute preventing the oxygen level to be too low. Besides the mix valve 207 described in this alternative embodiment the rebreathing system 1 works as described earlier so that if the oxygen supply from the oxygen supplying member 5 is stopped the valve arrangement 8 closes and the breathing passage 42 is also closed, preventing the user to inhale breathing gas where the CO₂ has been absorbed but no oxygen has been added which otherwise may cause the user to suffer from hypoxia that in the worst may be lethal.

The skilled person realises that the mix valve 207, can be both variable and arranged to have two or more fixed positions. In the preferred embodiment it may be variable or have different steps of variability, to achieve a desired oxygen saturation. The mix valve 207 may also be arranged at other places than described above and has other embodiments. This is an easy and cheap alternative to get control of the oxygen saturation. Also the control device 200 may be designed in other ways than described above, for example, the control device may be a knob or a button. It is foreseen that the possibility to completely shut off the counter lung 2 may be possible.

The valve arrangement 8 described above is intended to be used in any rebreathing system in which a carbon dioxide absorber is used as a protection against use of said system if the oxygen supply for some reason has stopped. The above described rebreathing system 1 has a variety of application fields e.g. it would be a great advantage to connect this rebreathing system 1 to a medical emergency oxygen supply system for use on board aircrafts. Today's oxygen supply circuits provide the patient with an oxygen addition, but the major part of the oxygen is then exhaled into the ambient air. If a rebreathing system 1 according to the invention is connected to this oxygen supply circuit the oxygen will last up to 3-4 times longer. Alternatively the stock of e.g. oxygen cylinders may be reduced, which will lead to both cost savings and savings concerning storage space.

The described rebreathing system 1 will also be beneficial in the treatment of divers suffering decompression illness. In this treatment a high inspiratory oxygen fraction during prolonged time is essential to facilitate elimination of nitrogen from the body after diving and/or during transport to a recompression chamber. With a rebreathing system according to the invention high oxygen fraction may be obtained, nitrogen may be vented off while at the same time prolonged use of the oxygen source is obtained in combination with safety against hypoxia.

Another possible application is to use the rebreathing system 1 in one-time rebreathers which makes it possible to use them in many different situations. A one-time rebreather preferably has a chemically bound oxygen source, e.g. per-chlorates which generate oxygen in a manner known per se. The rebreather may be enclosed in a vacuum pack or the like. When the vacuum pack is torn up, a mechanism e.g. a partition that at the tearing is ripped such that the chemical substances get into contact with each other, starting the oxygen producing reaction for the oxygen supply.

Today many smoke-helmeted firemen risk their lives since when they are in e.g. a smoke-filled house and their breathing equipment announce that it is time to go out, if they in that moment see or hear a person, they are doing everything to save this person often risking their own lives. If the firemen have some one-time rebreathers in their outfit they could fast and easy place the rebreather on the found person, run out and sending in a new ready fireman to get the person. It could also save persons found in a smoke-filled area if they get a rebreather directly instead of continuing breathing the smoke-filled air until evacuated.

A one-time rebreather could save lives if they are easy of access on strategically places. All people should have some in their homes, e.g. in the bedside table, so that at detection of fire or when the fire alarm is activated, its easy to grab a rebreather, tear up the pack (that starts the oxygen supply) and put it on and the person have about 15-20 min to find a way out without getting injured by possible smoke development.

A rebreathing system according to the invention would with advantage exist in e.g. first aid kits, in cars, so that the first person at an accident may put on a rebreather on injured persons giving them oxygen while waiting for ambulance. In big arenas there could be placed a rebreather under every seat, easy accessible for everyone if an accident occurs. Hotels often have high safety demands and with this one-time rebreather every room could be equipped with one.

The invention is not to be seen as limited by the embodiments described above but can be varied within the scope of the appended claims, as will be readily apparent to the person skilled in the art. For instance the breathing mask could instead of just cover the respiratory ways also cover the eyes and prevent getting e.g. smoke in the eyes. All the valves described above may be placed in other positions without departing from the inventive concept.

1-19. (canceled)

20. A valve arrangement arrangable to be used in a rebreathing system, said rebreathing system comprising an oxygen supplying member, a breathing mask, a gas reconditioning unit where carbon dioxide in the exhaled gas is absorbed, a counter lung and a breathing passage, wherein said valve arrangement is arrangable to be used in a two way breathing passage between said counter lung and said breathing mask of said rebreathing system, said valve arrangement containing an oxygen supply arrangement arranged to supply oxygen to the breathing passage provided that a predetermined level of oxygen pressure is exerted on said supply arrangement from said oxygen supplying member and arranged to close the breathing passage when said oxygen supply pressure is below said predetermined level.

21. The valve arrangement according to claim 20, wherein said supply means comprise an oxygen receiving chamber provided with an oxygen inflow and an oxygen outflow having an oxygen flow restrictor.

22. The valve arrangement according to claim 21, wherein a spring biased protective valve is in fluid communication with said oxygen receiving chamber, said oxygen supply pressure contained therein acting on said protective valve to set the protective valve in an open position.

23. The valve arrangement according to claim 22, wherein the spring biased protective valve is regulated to open at a pressure that is significantly lower than the oxygen supply pressure.

24. The valve arrangement according to claim 21, wherein said oxygen flow restrictor is a throttle valve, preferably a set screw.

25. The valve arrangement according to claim 22, wherein said spring biased protective valve comprises a breathing passage closing member co-acting with a seat (43, 143) in a protective valve housing.

26. The valve arrangement according to claim 25, wherein said seat is an integrated part of a connecting device between the breathing mask and said other parts of the rebreathing system.

27. The valve arrangement according to claim 25, wherein the protective valve comprises a protective valve housing comprising an outer end and an inner end, said inner end being placed inside the breathing passage, and said outer end facing the surroundings.

28. The valve arrangement according to claim 27, wherein said protective valve housing accommodates a piston connected to said breathing passage closing member via a piston rod passing, in an airtight manner, a through hole of an inner end wall of the protective valve housing, said breathing passage closing member preferably being a valve disc.

29. The valve arrangement according to claim 27, wherein the outer end is provided with at least one opening to the surroundings.

30. The valve arrangement according to claim 28, wherein said piston is arranged in sealed abutment to an inside of said protective valve housing.

31. A rebreathing system an oxygen supplying member arranged to be supplied with oxygen from an internal or external oxygen source, a breathing mask, a gas reconditioning unit where carbon dioxide in the exhaled gas is absorbed, a counter lung, a breathing passage between said counterlung and said breathing mask, wherein it comprises a valve arrangement according to claim 20.

32. The rebreathing system according to claim 31, wherein said oxygen supplying source supplies oxygen having an oxygen pressure in the range of 100-300 kPa, preferably 200-300 kPa.

33. The rebreathing system according to claim 31, wherein said oxygen supplying source is a pressurized oxygen gas source or a chemically bound oxygen source.

34. The rebreathing system according to claim 31, wherein it comprises a heat exchanger which forms part of the breathing passage, preferably constituting an integrated part of a wall of the breathing passage, said heat exchanger being arranged to absorb heat generated by the CO₂-absorbing reaction in the gas reconditioning unit.

35. The rebreathing system according to claim 34, wherein an upper end of the gas reconditioning unit is arranged at a relative angle to the extension of the heat exchanger wherein a compartment inside the heat exchanger having a volume in the order of max 0.5 l, preferably max 0.2 l, is formed.

36. The rebreathing system according to claim 35, wherein said heat exchanger constitutes a major part of the wall enclosing said compartment thereby providing a large contact area for the breathing gas.

37. The rebreathing system according to claim 31, wherein said breathing mask comprises an overpressure valve and an air inflow valve in order to secure free passage of surrounding air to and from the mask when the protective valve is in a closed position.

38. The rebreathing system according to claim 37, wherein said air inflow valve comprises a whistle.
